(19) United States
(12) Patent Application Publication
(10) Pub. No.: US 2025/0049374 A1
(43) Pub. Date: Feb. 13, 2025

(54) SYSTEMS AND METHODS FOR DETECTING CARDIAC PAUSE

(71) Applicant: Cardiac Pacemakers, Inc., St. Paul, MN (US)

(72) Inventors: Krzysztof Z. Siejko, Maple Grove, MN (US); Vasanth Ravikumar, Minneapolis, MN (US)

(21) Appl. No.: 18/798,424

(22) Filed: Aug. 8, 2024

Related U.S. Application Data
(60) Provisional application No. 63/531,346, filed on Aug. 8, 2023.

(51) Int. Cl. A61B 5/352; A61B 5/00; A61B 5/339; A61B 5/364; G16H 40/67

(52) U.S. Cl. CPC A61B 5/352; A61B 5/339; A61B 5/364; A61B 5/7221; G16H 40/67

(57) ABSTRACT

Systems and methods for verifying cardiac pause episodes detected by a medical device are disclosed. A medical-device system comprises a physiological event detector to receive information about a cardiac pause episode detected by a medical device from a patient using a device-based detection algorithm. The physiological event detector uses an algorithm different from the device-based detection algorithm to generate one or more metrics including at least one entropy metric from the received information about the cardiac pause episode, and verify a presence or absence of cardiac pause in the device-detected cardiac pause episode using such metrics. The verified presence or absence of the cardiac pause may be presented to a user or a process to determine a medical condition, such as a risk of syncope in the patient.

### CLAIM OF PRIORITY

This application claims the benefit of U.S. Provisional Application No. 63/531,346, filed on Aug. 8, 2023, which is hereby incorporated by reference in its entirety.

### TECHNICAL FIELD

This document relates generally to medical devices, and more particularly, to systems, devices and methods for detecting and verifying cardiac pause.

### BACKGROUND

Cardiac pause generally refers to an absence of cardiac electrical activity for an extended period of time. The electrocardiogramepresentation of cardiac pause includes a prolonged R-R interval representing an interruption in ventricular depolarization. Cardiac pause may be attributed to sinus node dysfunction (sinus arrest), or atrioventricular conduction disturbance or block, among others. For example, sinus arrest occurs when the sinoatrial node transiently ceases to generate the electrical impulses that normally stimulate the myocardial tissues to contract and thus the heart to beat. Cardiac pause may cause severe lightheadedness, dizziness, near syncope, or a syncopal or passing-out episode. Cardiac pause, among other cardiac arrhythmias, have been found to be associated with sleep apnea.

Ambulatory medical devices (AMDs), such as pacemakers, implantable defibrillators, implantable cardiac resynchronization therapy devices, or insertable cardiac monitors, have been used for monitoring patient health condition or disease states. Some AMDs are able to provide therapies to treat or alleviate certain health conditions. For example, implantable cardioverter-defibrillators may be used to monitor for certain abnormal heart rhythms, such as bradycardia or tachycardia, and to deliver electrical energy to the heart to correct the abnormal rhythms. Some AMDs can monitor chronic worsening of cardiac hemodynamic performance, such as due to congestive heart failure (CHF), and to provide cardiac stimulation therapies, including cardiac resynchronization therapy (CRT) to correct cardiac dyssynchrony within a ventricle or between ventricles.

### OVERVIEW

Conventional cardiac pause detection uses R-R intervals measured from electrocardiogramar subcutaneous or intracardiac electrograms (EGMs) recorded from a patient. A cardiac pause episode can be detected based on a prolongation of R-R intervals exceeding a specific pause duration. The R-R interval based cardiac pause detection may be affected by noise or artifacts (e.g., motion artifacts), undersensing due to low signal amplitude, or flatline or missing data. For example, implant noise (no R waves present, or non-physiological signals recorded), change in amplitude of R-wave amplitudes (especially in low amplitude cases) such as in the presence of premature ventricular contraction (PVC), chronic undersensing with PVC, chronic undersensing with large artifacts, or noisy ECG or EGMs particularly prior to the pause segment (noisy pre-pause segment) may cause false positive detections of cardiac pause. The false positive detections may lead to inappropriate therapies or unnecessary interventions, reduce efficiency of resource usage, consume more battery power and resources, and reduce battery life of a medical device.

A patient management system can manage a large volume of data associated with physiological events detected and recorded by AMDs. The device-recorded event data may be stored in the patient management system. A clinician may review the event data, make diagnosis or adjudication of the device-detected events, schedule patient follow-up visits, adjust device therapy or prescribe further treatment, etc. With a large number of devices connected to and communicating with the patient management system, reviewing such large volume of device-recorded event data generally take significant amount of time and resources, and can be costly for a healthcare facility. On the other hand, due to the limitations of the event detection algorithms used by the AMDs and the power and resource constraints, inappropriate event detections (e.g., false positive events) may exist. Some patients may have more repetitive false positive detections than others. The false positive event episodes increase the clinician's burden and reduce the efficiency of the event review process.

The present inventors have recognized an unmet need for apparatus and techniques to automate the event review process by reducing the false positive event episodes to be reviewed by a human expert. Although the present document focuses on device-detected cardiac pause episodes, other event episodes detected by AMDs have also been considered. The reduction of false positive cardiac pause episodes can be achieved using improved offline pause verification techniques that may be implemented in a patient management system, such as a server operatively in communication with AMDs. In accordance with an embodiment, a medical-device system comprises a physiological event detector to receive information about a cardiac pause episode detected by a medical device from a patient using a device-based detection algorithm. The physiological event detector can use a verification algorithm, different from the device-based detection algorithm, to generate one or more metrics, including at least one entropy metric, from the received information about the cardiac pause episode, and verify a presence or absence of cardiac pause in the device-detected cardiac pause episode using the one or more metrics. The verified presence or absence of the cardiac pause may be presented to a user or a process executable by the medical-device system to determine a medical condition, such as a risk of syncope, in the patient.

Example 1 is a medical-device system for verifying cardiac pause episodes detected by a medical device in a patient. The system may include a cardiac pause verification circuit configured to: receive information about a device-detected cardiac pause episode detected by the medical device using a device-based detection algorithm; generate one or more metrics, including at least one entropy metric, from the received information about the device-detected cardiac pause episode; and verify a presence or absence of cardiac pause in the device-detected cardiac pause episode using the generated one or more metrics and a verification algorithm different from the device-based detection algorithm; and an output circuit configured to provide the verified presence or absence of the cardiac pause to a user or a process executable by the medical-device system.

In Example 2, the subject matter of Example 1 optionally includes, the received information about the device-detected cardiac pause episode that may include a cardiac signal recorded by and stored in the medical device.

In Example 3, the subject matter of Example 2 optionally includes the received information about the device-detected cardiac pause episode that may further include event markers generated by and stored in the medical device.

In Example 4, the subject matter of Example 3 optionally includes the event markers that may include a pause onset marker marking a beginning of the device-detected cardiac pause episode and a pause end marker marking an end of the device-detected cardiac pause episode, the pause onset marker and the pause end marker partitioning the cardiac signal into a pre-pause segment prior to the pause onset marker, an inner pause segment between the pause onset marker and the pause end marker, and a post-pause segment following the pause end marker.

In Example 5, the subject matter of Example 4 optionally includes the event markers that may further include type or timings of heart beats or noise events in the cardiac signal recognized by the medical device.

In Example 6, the subject matter of any one or more of Examples 4-5 optionally includes the at least one entropy metric that may include a Shannon entropy of the inner pause segment of the cardiac signal, wherein the cardiac pause verification circuit is configured to verify the presence or absence of cardiac pause in the device-detected cardiac pause episode using the Shannon entropy of the inner pause segment.

In Example 7, the subject matter of Example 6 optionally includes the one or more metrics that may include a representative amplitude of cardiac events in the pre-pause segment, wherein the cardiac pause verification circuit is configured to verify the presence or absence of cardiac pause in the device-detected cardiac pause episode further using the representative amplitude of cardiac events in the pre-pause segment.

In Example 8, the subject matter of any one or more of Examples 4-7 optionally includes the at least one entropy metric that may include a sample entropy of the inner pause segment of the cardiac signal, wherein the cardiac pause verification circuit is configured to verify the presence or absence of cardiac pause in the device-detected cardiac pause episode using the sample entropy.

In Example 9, the subject matter of Example 8 optionally includes the one or more metrics that may include a first amplitude ratio of a representative cardiac event amplitude in the pre-pause segment to a representative amplitude of the inner pause segment, wherein the cardiac pause verification circuit is configured to verify absence of cardiac pause in the device-detected cardiac pause episode further using the first amplitude ratio.

In Example 10, the subject matter of any one or more of Examples 4-9 optionally includes the cardiac pause verification circuit configured to verify the presence or absence of cardiac pause in the device-detected cardiac pause episode further using one or more of: a count of noise events detected by the medical device from a specific portion of the pre-pause segment; an average heart rate in the pre-pause segment; or a second amplitude ratio of a representative amplitude of cardiac events in the post-pause segment to a representative amplitude of the inner pause segment.

In Example 11, the subject matter of any one or more of Examples 4-10 optionally includes the cardiac signal that may include an electrocardiogram (ECG), the device-based detection algorithm includes a first R wave detector, the verification algorithm includes a second R wave detector different from and more sensitive than the first R wave detector in detecting R wave timings in the ECG, wherein the cardiac pause verification circuit is configured to verify the presence or absence of cardiac pause in the device-detected cardiac pause episode further based on a comparison of timings of R waves detected by the second R wave detector and timings of R waves detected by the first R wave detector.

In Example 12, the subject matter of any one or more of Examples 1-11 optionally includes the verification algorithm that may include a decision tree taking the one or more metrics as decision nodes, or a neural network model taking the one or more metrics as input layer parameters.

In Example 13, the subject matter of any one or more of Examples 1-12 optionally includes an ambulatory medical device (AMD) and an external system operatively in communication with the AMD, wherein the AMD includes circuitry configured to sense physiological information in a patient, to detect the cardiac pause episode from the sensed physiological information, and to store the information about the detected cardiac pause episode, wherein the external system including the cardiac pause verification circuit.

In Example 14, the subject matter of any one or more of Examples 1-13 optionally includes the output circuit configured to: present the information about the device-detected cardiac pause episode on a display in response to the verified presence of cardiac pause in the device-detected cardiac pause episodes; and withhold display of the information about the device-detected cardiac pause episode in response to the verified absence of cardiac pause in the device-detected cardiac pause episodes.

In Example 15, the subject matter of any one or more of Examples 1-14 optionally includes an event detector circuit configured to determine a risk of syncope in the patient based at least in part on the verified presence or absence of the cardiac pause.

Example 16 is a method of verifying cardiac pause episodes detected by a medical device in a patient, the method includes steps of: receiving information about a device-detected cardiac pause episode detected by the medical device using a device-based detection algorithm; generating one or more metrics, including at least one entropy metric, from the received information about the device-detected cardiac pause episode; verifying a presence or absence of cardiac pause in the device-detected cardiac pause episode using the generated one or more metrics and a verification algorithm different from the device-based detection algorithm; and providing the verified presence or absence of the cardiac pause to a user or a process.

In Example 17, the subject matter of Example 16 optionally includes the received information about the device-detected cardiac pause episode that may include a cardiac signal and event markers generated by and stored in the medical device.

In Example 18, the subject matter of Example 17 optionally includes the event markers that may include a pause onset marker marking a beginning of the device-detected cardiac pause episode, and a pause end marker marking an end of the device-detected cardiac pause episode, the pause onset marker and the pause end marker partitioning the cardiac signal into a pre-pause segment prior to the pause onset marker, an inner pause segment between the pause onset marker and the pause end marker, and a post-pause segment following the pause end marker.

In Example 19, the subject matter of Example 18 optionally includes the at least one entropy metric that may include a Shannon entropy of the inner pause segment of the cardiac signal, the one or more metrics include a representative amplitude of cardiac events in the pre-pause segment, wherein verifying the presence or absence of cardiac pause in the device-detected cardiac pause episode includes using the Shannon entropy of the inner pause segment and the representative amplitude of cardiac events in the pre-pause segment.

In Example 20, the subject matter of any one or more of Examples 18-19 optionally includes the at least one entropy metric that may include a sample entropy of the inner pause segment of the cardiac signal, the one or more metrics include a first amplitude ratio of a representative cardiac event amplitude in the pre-pause segment to a representative amplitude of the inner pause segment, wherein verifying the presence or absence of cardiac pause in the device-detected cardiac pause episode includes using the sample entropy of the inner pause segment and the first amplitude ratio.

In Example 21, the subject matter of any one or more of Examples 18-20 optionally includes verifying the presence or absence of cardiac pause in the device-detected cardiac pause episode that may include using one or more of: a count of noise events detected by the medical device from a specific portion of the pre-pause segment; an average heart rate in the pre-pause segment; or a second amplitude ratio of a representative amplitude of cardiac events in the post-pause segment to a representative amplitude of the inner pause segment.

In Example 22, the subject matter of any one or more of Examples 18-21 optionally includes he cardiac signal that may include an electrocardiogram (ECG), the device-based detection algorithm includes a first R wave detector, the verification algorithm includes a second R wave detector different from and more sensitive than the first R wave detector in detecting R wave timings in the ECG, wherein verifying the presence or absence of cardiac pause in the device-detected cardiac pause episode is based at least in part on a comparison of timings of R waves detected by the second R wave detector and timings of R waves detected by the first R wave detector.

In Example 23, the subject matter of any one or more of Examples 16-22 optionally include: presenting the information about the device-detected cardiac pause episode on a display in response to the verified presence of cardiac pause in the device-detected cardiac pause episodes; and withholding display of the information about the device-detected cardiac pause episode in response to the verified absence of cardiac pause in the device-detected cardiac pause episodes.

The cardiac pause detection techniques as described in this document may be implemented in an external system such as a communicator, mobile monitor, programmer, or a remote patient management system in communication with AMDs. In an example, device-detected cardiac pause episodes may be reviewed by a clinician for further diagnosis. Reviewing and processing device-detected cardiac pause episodes with mixed or unidentified certainties may put a high demand for time, effort, and resources, and can be costly for a healthcare facility. The offline, server-based pause algorithm may improve the sensitivity and specificity for detecting or verifying cardiac pause episodes. By excluding the false positive episodes from the pool of events for review, clinicians' burden can be reduced, efficiency of event review process can be improved, and the overall healthcare cost associated with false positive pause detections can be reduced. Additionally, the improvements in cardiac pause detection may be achieved with little to no additional cost or added system complexity. In some examples, existing system performance may be maintained (e.g., high arrhythmia detection sensitivity and specificity, etc.) using lower cost or less obtrusive systems, apparatus, and methods. With improved cardiac pause verification or verification, fewer alerts to the clinician are provided, fewer unnecessary drugs and procedures may be scheduled, prescribed, or provided, and an overall system cost and power savings may be realized in contrast to existing medical devices and systems. Consequently, medical resources may be better aligned to serve more patients, and the patient management cost in a healthcare facility may be reduced.

This Overview is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates an example of a cardiac pause episode detected by an implantable medical device in a patient.

FIGS. 3A-3B illustrate examples of appropriately detected and inappropriately detected cardiac pause episodes that are detected by and stored in a medical device.

FIG. 4 illustrates generally an example of a cardiac pause verification system for verifying cardiac pause episodes detected by a medical device in a patient.

FIG. 5 illustrates examples of signal metrics that may be generated from a device-detected cardiac pause episode.

FIGS. 6A-6E illustrate example methods of verifying a presence or absence of cardiac pause in a device-detected cardiac pause episode using one or more signal metrics as shown in FIG. 5.

FIG. 7 is a flowchart illustrating an example of a method of verifying cardiac pause episodes detected by a medical device in a patient.

FIG. 8 illustrates generally a block diagram of an example machine upon which any one or more of the techniques (e.g., methodologies) discussed herein may perform.

### DETAILED DESCRIPTION

Disclosed herein are systems, devices, and methods for verifying cardiac pause episodes detected by a medical device. An exemplary medical-device system comprises a physiological event detector to receive information about a cardiac pause episode detected by a medical device from a patient using a device-based detection algorithm. The physiological event detector can use a verification algorithm, different from the device-based detection algorithm, to generate one or more metrics including at least one entropy metric from the received information about the cardiac pause episode, and verify a presence or absence of cardiac pause in the device-detected cardiac pause episode based at least in part on the one or more metrics. The verified presence or absence of the cardiac pause may be presented to a user or a process executable by the medical-device system, such as for determining the patient's risk of syncope.

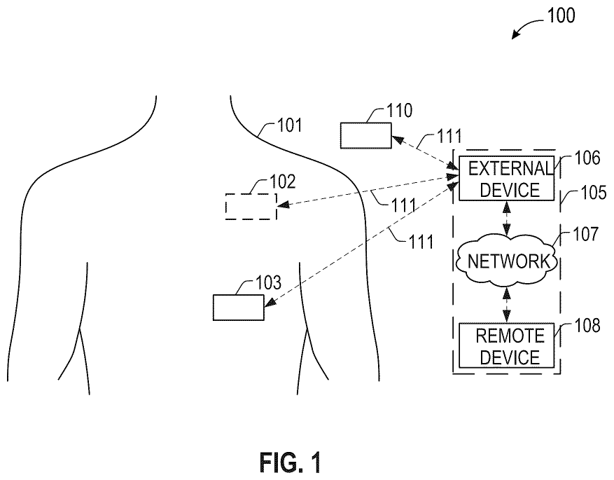

FIG. 1 illustrates an example patient management system 100 and

portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more ambulatory medical devices, an external system 105, and a communication link 111 providing for communication between the one or more ambulatory medical devices and the external system 105. The one or more ambulatory medical devices can include an implantable medical device (IMD) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

The IMD 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. Examples of the detected conditions or diseases may include cardiac arrhythmias of various types, including cardiac pause episodes characterized by an absence of cardiac electrical activity in for an extended period of time. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the IMD 102 or one or more of the other ambulatory medical devices, etc. In some examples, therapy can include cardiac resynchronization therapy for rectifying dyssynchrony and improving cardiac function in heart failure patients. In other examples, the IMD 102 can include a drug delivery system, such as a drug infusion pump to deliver drugs to the patient for managing arrhythmias or complications from arrhythmias, hypertension, or one or more other physiologic conditions. In other examples, the IMD 102 can include one or more electrodes configured to stimulate the nervous system of the patient or to provide stimulation to the muscles of the patient airway, etc.

The WMD 103 can include one or more wearable or external medical sensors or devices (e.g., automatic external defibrillators (AEDs), Holter monitors, patch-based devices, smart watches, smart accessories, wrist-or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This can include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information can include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radiofrequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected physiological event (e.g., detected by one or more ambulatory medical devices such as the IMD 102 or the WMD 103) can be prioritized using a similarity metric between the physiological data associated with the detected physiological event to physiological data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible. One or more of the external device 106 or the remote device 108 can

output the detected physiological events to a system user (e.g., display on a user interface), such as the patient or a clinician, or to a process including, for example, an instance of a computer program executable in a microprocessor. In some examples, one or more of the external device 106 or the remote device 108 can include circuitry to perform analysis of the device-detected physiological event (e.g., a cardiac pause episode), and verify whether or not the detected physiologic event is indeed present. A verified presence indicates that the device-detected physiological event is a “true positive” event. A verified absence indicates that the device-detected physiological event is a “false positive” event. The external device 106 or the remote device 108 may output the detected physiological events to the system user based on a result of verification. In an example, only the “true positive” episodes are automatically output to the system user. To reduce the review burden, the “false positive” episodes are not automatically presented to the user, but may be provided upon the user's request.

In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

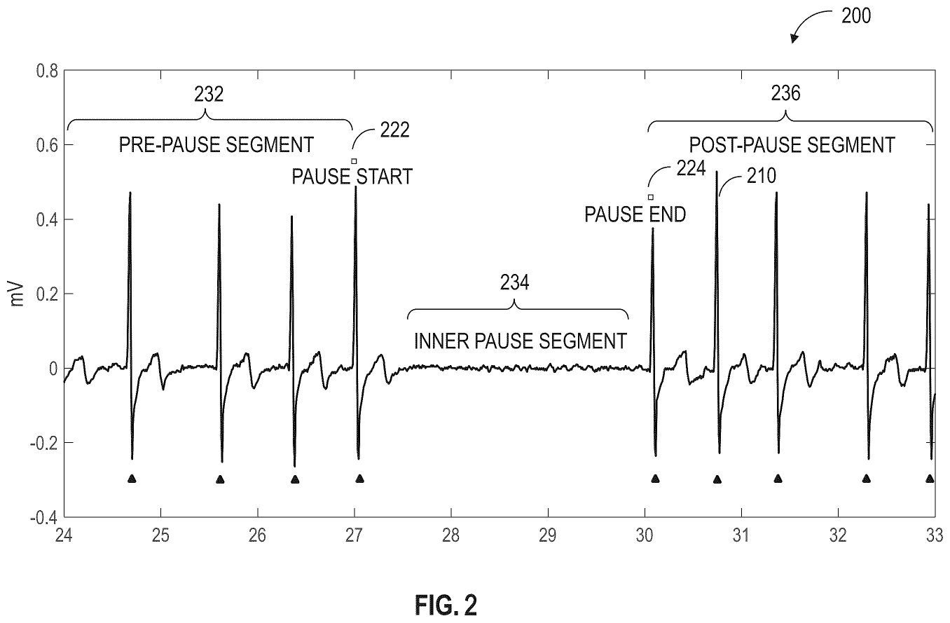

FIG. 2 illustrate an example of a cardiac pause episode 200 detected by an implantable medical device in a patient. The implantable medical device can sense physiological information such as a cardiac signal in a patient, and detect cardiac pause episodes using a device-based detection algorithm using amplitudes of ventricular depolarizations, such as intensity of the R waves or the QRS complexes detected from an electrocardiogram (ECG). A cardiac pause episode is detected when the time interval between two consecutive ventricular depolarizations (e.g., an R-to-R interval) exceeds a specified pause threshold. In some examples, signal-to-noise ratio of certain portions of the ECG may be checked to ensure the pause detection is not interfered or misled by noise.

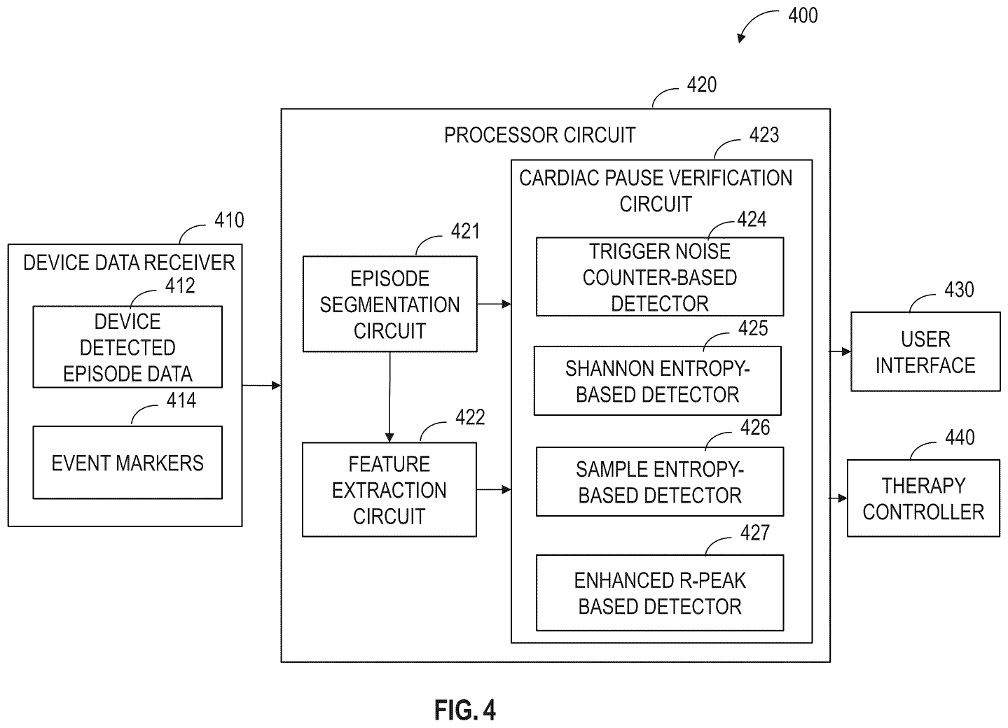

In response to the detection of a cardiac pause, the implantable medical device may record a cardiac signal 210 representative of ventricular depolarizations before, during, and after the detected cardiac pause. The implantable medical devices may additionally generate event markers identifying the type or the timing of certain events (e.g., R wave peak in the cardiac signal). Examples of the event markers include a pause onset marker 222 (“pause start” as annotated in FIG. 2) marking a beginning in time of the device-detected cardiac pause episode, and a pause end marker 224 (“pause end” as annotated in FIG. 2) marking an end in time of the device-detected cardiac pause episode. The pause onset marker 222 and the pause end marker 224 may be used to partition the recorded cardiac signal 210 into a pre-pause segment 232 prior to the pause onset marker 222 for a specific duration (e.g., up to 30 seconds before the pause onset marker 222 in an example), an inner pause segment 234 between the pause onset marker 222 and the pause end marker 224, and a post-pause segment 236 following the pause end marker 224 for a specific duration (e.g., up to 30 seconds after the pause end marker 224 in one example). The event markers may also include type or timings of heart beats or noise events in the cardiac signal recognized by the medical device, including, for example, R wave timing markers for a number of R waves detected in the pre-pause segment (e.g., preR0, preR1, preR2, and preR3 as annotated in FIG. 2) and in the post-pause segment (e.g., postR1, postR2, postR3, and postR4 as annotated in FIG. 2). As will be discussed further below such with respect to FIG. 4, a number of signal metrics may be generated using the event markers and one or more of the pre-pause segment 232, the inner pause segment 234, or the post-pause segment 236 of the recorded cardiac signal 210. Such signal metrics can be used for verifying a presence or absence of cardiac pause in the device-detected cardiac pause episodes.

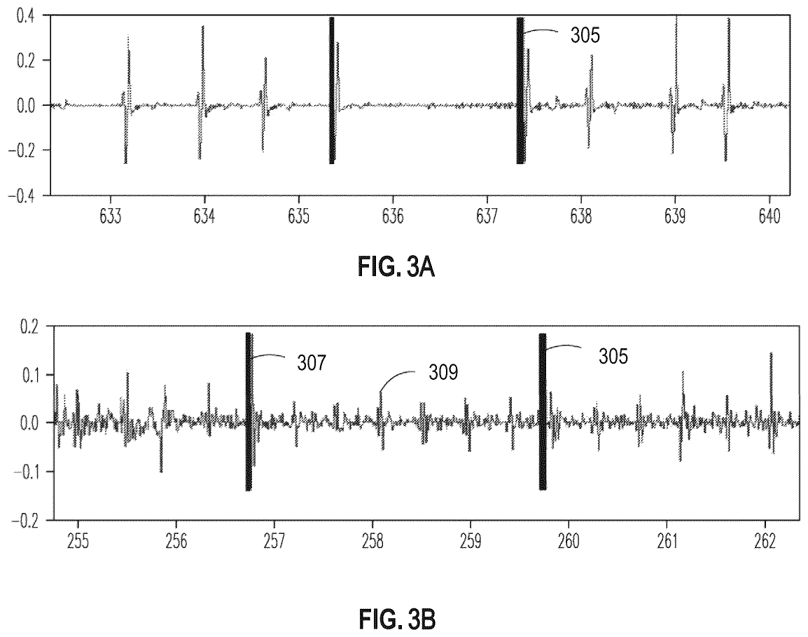

Due to the constraints of battery power and computational resources in an AMD, simple device-based pause detection algorithms may cause inappropriately detected, or false positive, cardiac pause episodes. One cause of the false positive detections is due to undersensing caused by physiological R wave amplitude drop. FIGS. 3A-3B illustrate examples of appropriately detected and inappropriately detected cardiac pause episodes. FIG. 3A shows an appropriately detected episode of cardiac pause, characterized by temporary cessation of sinus node discharges and electrocardiogramted by an absence of P waves (atrial depolarization) and associated QRS complex (ventricular depolarization) in an electrocardiogram (ECG). The graph includes a pause marker 305 to indicate the cardiac pause. As shown in FIG. 3A, the signal waveform is essentially a flat line between the last R-wave before the pause and the marker. FIG. 3B shows a falsely detected cardiac pause episode due to undersensing caused by (physiological) R wave amplitude drop. The pause marker 305 indicates when cardiac pause was incorrectly declared. The large R-wave 307 before the pause marker raised the dynamic detection threshold. The subsequent R-waves of smaller amplitude 309 are missed due to the latency in the adjustment of the dynamic threshold causing the detection of a false cardiac pause. In addition to R wave undersensing, other causes of inappropriate pause detections may include implant noise (no R waves present, or non-physiological signals recorded), change in amplitude of R-wave amplitudes (especially in low amplitude cases) such as in the presence of premature ventricular contraction (PVC), chronic undersensing with PVC, chronic undersensing with large artifacts, or noisy ECG or EGMs particularly prior to the pause segment (noisy pre-pause segment), among others.

FIG. 4 illustrates generally an example of a cardiac pause verification system 400 for verifying device-detected cardiac pause episodes in a patient. The cardiac pause verification system 400 may include one or more of a device data receiver 410, a processor circuit 420, a user interface 430, and an optional therapy controller 440. Portions of the cardiac pause verification system 400 may be implemented in a patient management system, such as the external device 106 or the remote device 108 of the external system 105.

The device data receiver 410 may receive information stored in a medical device (e.g., IMD 102 or WMD 103 as shown in FIG. 1) about a cardiac pause episode detected by the medical device. In some examples, the device data receiver 410 may receive such information from a storage device such as an electronic medical record system that stores physiological information collected from the patient. The stored information may include device-detected episode data 412 and event marker 414. The episode data 412 may include a physiological signal sensed in response to the detection of a cardiac pause event, such as via one or more implantable, wearable, or otherwise ambulatory sensors or electrodes associated with the patient. Examples of the physiologic signal may include cardiac signals such as surface electrocardiogramaubcutaneous ECG, or intracardiac electrogram (EGM). Additionally or alternatively, the physiologic signal may include thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal such as sensed by an ambulatory accelerometer or acoustic sensors, physiologic response to activity, apnea hypopnea index, one or more respiration signals such as a respiration rate signal or a tidal volume signal, brain natriuretic peptide (BNP), blood panel, sodium and potassium levels, glucose level and other biomarkers and bio-chemical markers, among others. The event markers 414, as described above with respect to FIG. 2, may identify the type or timing of certain events (e.g., R wave peak in the cardiac signal), including a pause onset marker and pause end marker marking respectively the beginning and the end of the device-detected cardiac pause episode, type or timings of heart beats or noise events in the cardiac signal recognized by the medical device, among others.

The processor circuit 420, coupled to the device data receiver 410, may verify a presence or absence of cardiac pause in a device-detected cardiac pause episode using the device data about the device-detected pause episodes. The processor circuit 420 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The processor circuit 420 may include circuit sets comprising one or more other circuits or sub-circuits, including an episode segmentation circuit 421, a feature extraction circuit 422, and a cardiac pause verification circuit 423. The verification circuit 423 may further include a trigger noise counter-based detector 424, a Shannon entropy-based detector 425, a sample entropy-based detector 426, and an enhanced R peak based detector 427. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The episode segmentation circuit 421 may partition the recorded cardiac signal for the device-detected pause episode into different segments, including a pre-pause segment, an inner pause segment, and a post-pause segment using pause onset marker and pause end marker from the received event markers 414. As described above with respect to FIG. 2, the pre-pause segment may be defined as the segment before the pause onset marker, the inner pause segment may be defined as the portion of the cardiac signal between the pause onset marker and the pause end marker, the post-pause segment may be defined as the segment after the pause end marker.

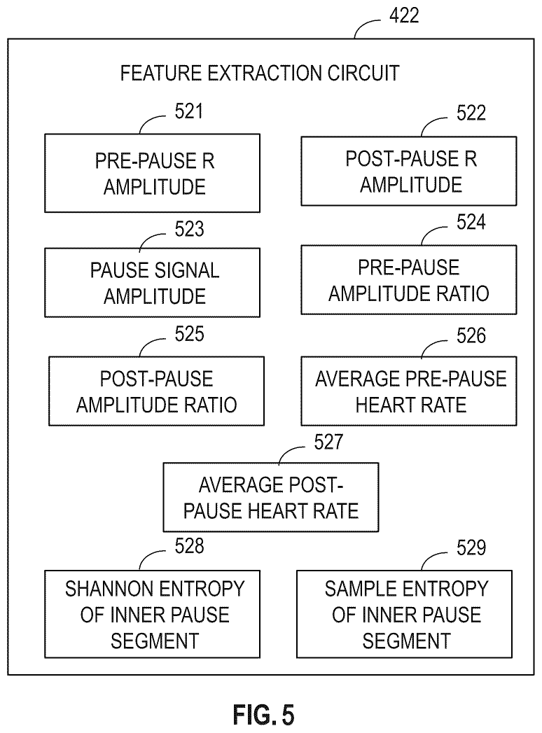

The feature extraction circuit 422 may generate one or more signal metrics using the device-detected episode data 412 and the event markers 414. In an example, the one or more signal metrics may be generated from one or more of the pre-pause segment, the inner pause segment, or the post-pause segment. Referring to FIG. 5, by way of example and not limitation, the signal metrics may include one or more of: (a) a representative pre-pause R wave amplitude, also referred to as pre-pause R amplitude 521, which may be computed as an average amplitude of a specific number N (e.g., three to five) R-waves before the pause onset marker (i.e., the last N R-waves in the pre-pause segment); (b) a representative post-pause R wave amplitude, also referred to as post-pause R amplitude 522, which may be computed as an average amplitude of a specific number M (e.g., three to five) R-waves after the pause end marker (i.e., the first M R-wave in the post-pause segment); (c) a representative inner pause signal amplitude 523, which can be computed as a specific percentile (e.g., 98^{th} percentile) signal amplitude of the absolute of amplitudes present in the inner pause segment; (d) a pre-pause amplitude ratio 524, computed as a ratio of a representative cardiac event amplitude in the pre-pause segment (e.g., the pre-pause R amplitude as described above) to the representative inner pause signal amplitude; (e) a post-pause amplitude ratio 525, computed as a ratio of a representative cardiac event amplitude in the post-pause segment (e.g., the post-pause R amplitude as described above) to the representative inner pause signal amplitude; (f) an average pre-pause heart rate 526, computed as the average heart rate using the device-detected heart beats (e.g., R waves) in the pre-pause segment and stored as event timing markers in the medical device; (g) an average post-pause heart rate 527, computed as the average heart rate using the device-detected heart beats (e.g., R waves) in the post-pause segment; (h) a Shannon entropy of the inner pause segment 528; or (i) a Sample entropy of the inner pause segment 529.

Shannon entropy measures the average amount of information conveyed by identifying the outcome of a random trial. It measures an irregularity or complexity of a signal or system. A smaller Shannon entropy indicates a lower degree of irregularity or a lower degree of complexity. A larger Shannon entropy indicates a higher degree of irregularity or a higher degree of complexity. Let X be the time series signal X=[x1, x2, . . . , xn] and p(x) is the probability density function of the amplitudes of the time series signals. The Shannon entropy H(X) of the time series X can be computed using Equation (1) below:

$$H ( X ) = - {\sum }_{x ∈ X} p ( x ) {log}_{2} p ( x ) \qquad ( 1 )$$

To calculate the Shannon entropy of the inner pause segment 528, a histogram of each data sample of the inner pause segment of the cardiac signal can be generated with a specified number M (e.g., 1000 in an example) equally spaced bins between the minimum and the maximum signal amplitudes. For each bin i, a probability p(i) may be calculated as a ratio of the sample counts in bin i, N(i), to the total number samples (L) in the signal, that is, p(i)=N(i)/L. The probability p(i) takes a value between zero and one. The Shannon entropy of the inner pause segment 528 can then be calculated according to Equation (1) above, that is, H(X)=Σᵢ₌₁^{M}p(i) log₂p(i).

Similar to Shannon entropy, sample entropy (SampEn) is also a measure of irregularity or complexity of a signal or system complexity. A smaller SampEn indicates a lower degree of irregularity or a lower degree of complexity. A larger SampEn indicates a higher degree of irregularity or a higher degree of complexity. Sample entropy is a modification of approximate entropy (ApEn) and has been widely used in physiological time series analysis. Let X be the time series signal X=[x1, x2, . . . , xn]. Templates can be formed in two dimensions: (i) templates (Xm) at m-th dimension (e.g., m=3), including [x1, x2, x3], [x2, x3, x4] . . . [xn−2, xn−1, xn]; and (ii) Templates (Xm+1) at (m+1)-th dimension (e.g, m+1=4), including, [x1,x2, x3, x4], [x2, x3, x4, x5] . . . [xn−3, xn−2, xn−1, xn]. Then, a Euclidean distance may be computed between all the templates at their respective dimensions, as given by Equations (2) and (3) below (where “sqrt” is a square root operator):

$${d}_{m} ( p , q ) = sqrt [ {( {p}_{1} - {q}_{1} )}^{2} + {( {p}_{2} - {q}_{2} )}^{2} + … + {( {p}_{m} - {q}_{m} )}^{2} ] where p , q ∈ {X}_{m} \qquad ( 2 )$$

$${d}_{m + 1} ( p , q ) = sqrt [ {( {p}_{1} - {q}_{1} )}^{2} + {( {p}_{2} - {q}_{2} )}^{2} + … + {( {p}_{m + 1} - {q}_{m + 1} )}^{2} ] where p , q ∈ {X}_{m + 1} \qquad ( 3 )$$

A threshold (r) for comparing the measured distances can be set. By way of example and not limitation, the threshold r can be set to a fraction (e.g., 0.2) of the standard deviation of the signal X. The fraction can be a programmable parameter. Defining the number of template pairs meeting the threshold requirement:

$$A = number of template pairs having {d}_{m + 1} ( p , q ) < r \qquad ( 4 )$$

$$B = number of template pairs having {d}_{m} ( p , q ) < r \qquad ( 5 )$$

Then, the SampEn can be computed as the negative of the natural logarithm (ln) of the ratio A/B, as given in Equation (6) below:

$$SampEn ( X ) = - ln ( A / B ) \qquad ( 6 )$$

Sample entropy of the inner pause segment 529 can be computed as described above using samples of the inner pause segment of the cardiac signal.

Referring back to FIG. 4, the cardiac pause verification circuit 423 can verify a presence or absence of cardiac pause in the device-detected cardiac pause episode using an algorithm different from the device-based detection algorithm used by the medical device in detecting in real time the cardiac pause episode. In an example, the algorithm used by the cardiac pause verification circuit 423 may include a decision tree that takes the one or more metrics as decision nodes. In another example, the algorithm used by the cardiac pause verification circuit 423 may include a neural network model, or other types of machine learning (ML) model, that takes the one or more metrics as input layer parameters. The decision tree, the neural network model, or other ML models may be trained to produce desirable pause verification or verification performance in a training dataset of device-detected pause episodes.

In the example as illustrated in FIG. 4, the algorithm used by the cardiac pause verification circuit 423 may include a plurality of detectors, including, for example, a trigger noise counter-based detector 424, a Shannon entropy-based detector 425, a sample entropy-based detector 426, or an enhanced R-peak based detector 427. Each of these detectors is capable of detecting or verifying a presence or absence of cardiac pause using one or more signal metrics generated by the feature extraction circuit 422, such as those described above with respect to FIG. 5.

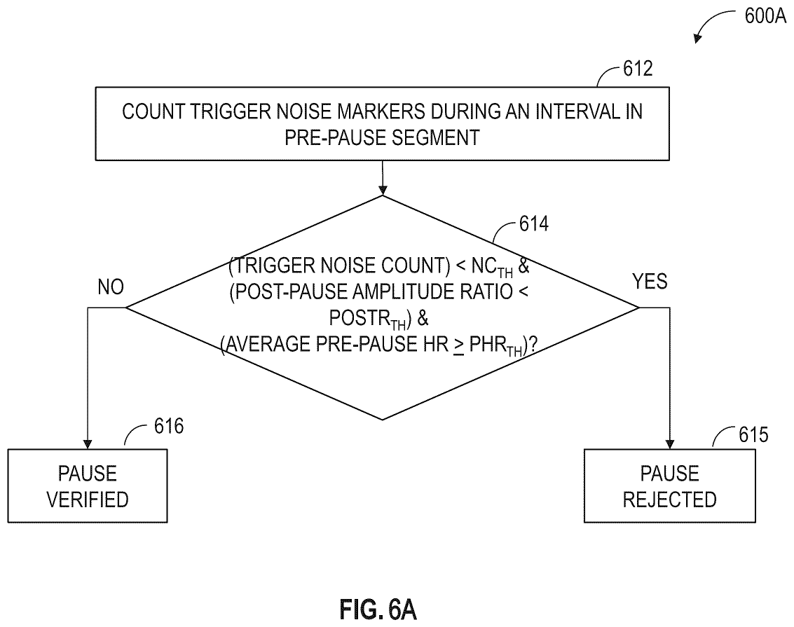

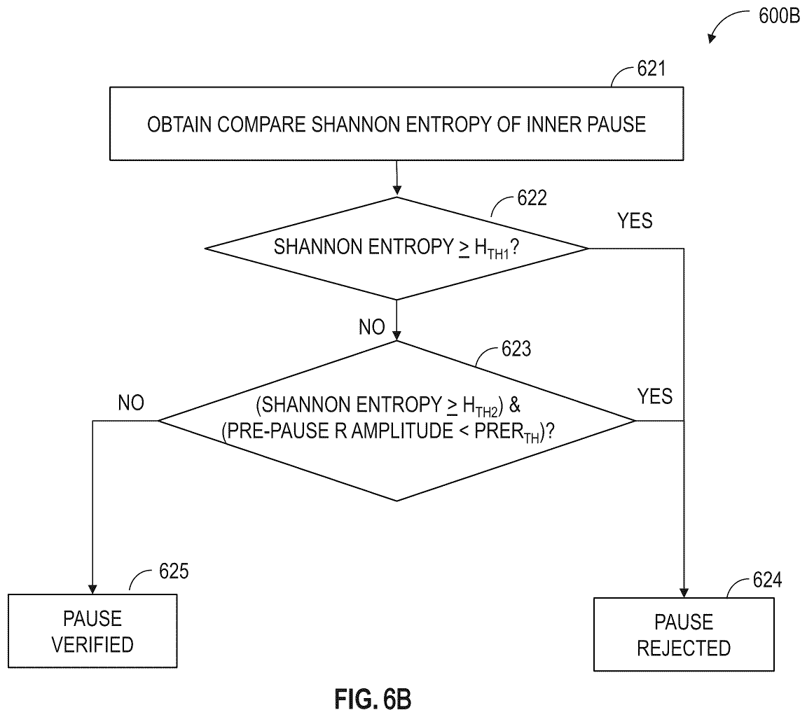

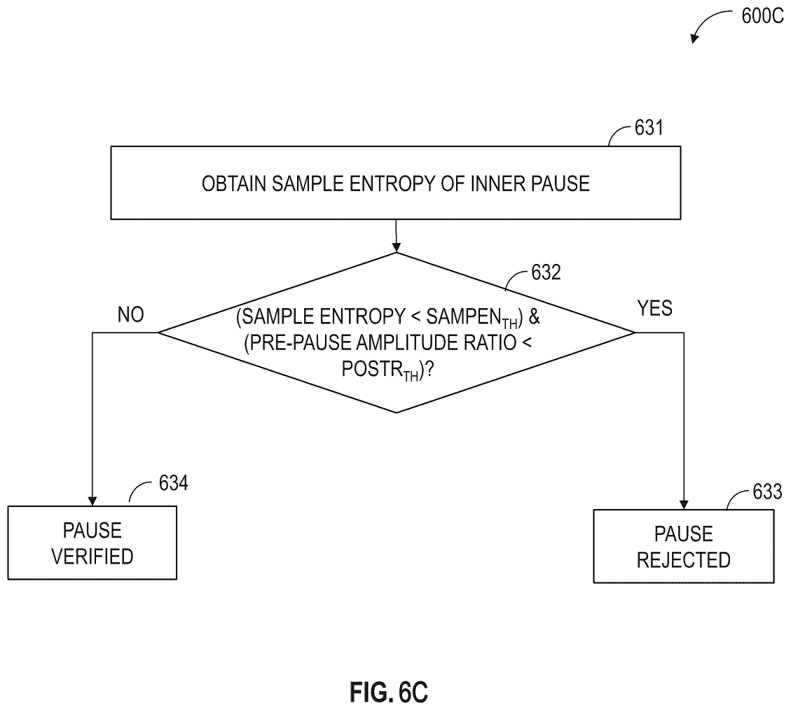

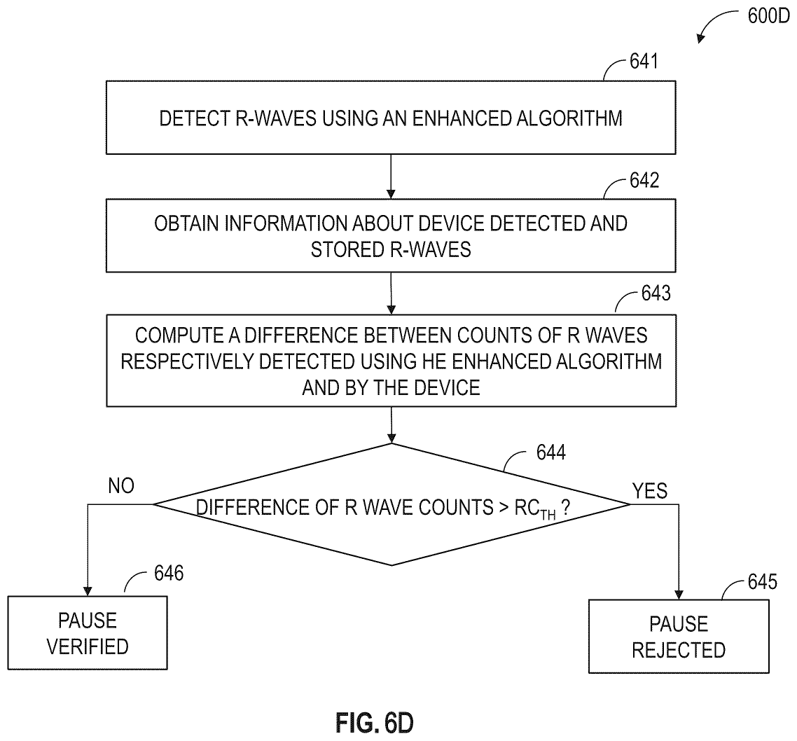

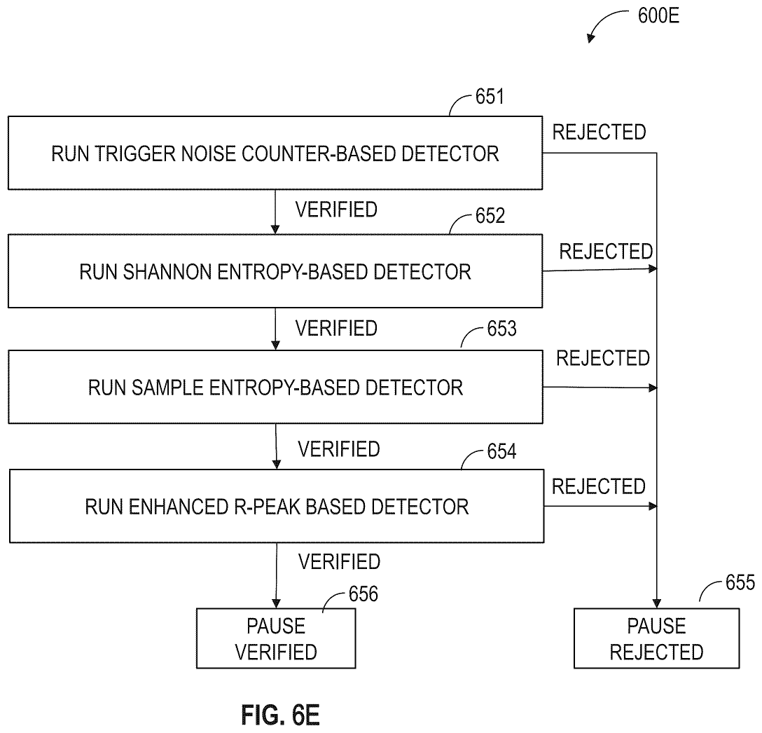

The trigger noise counter-based detector 424 can verify a presence or absence of cardiac pause using device-detected noise information in a portion of the pre-pause segment of the cardiac signal. Such noise information may be stored as trigger noise markers in the event markers 414 and received by the device data receiver 410. In an example, the trigger noise counter-based detector 424 can count the trigger noise markers during an interval defined by last N R-waves in the pre-pause segment, such as between PreR0 to PreR3 as shown in FIG. 2. The trigger noise counter-based detector 424 may additionally use certain metrics generated by the feature extraction circuit 422, including the post-pause amplitude ratio 525 and average pre-pause heart rate 526. FIG. 6A is a flow chart of an example pause verification algorithm 600A that may be used by the trigger noise counter-based detector 424. At 612, the trigger noise markers are counted during a specific interval in the pre-pause segment. At 614, the trigger noise count, the post-pause amplitude ratio, and the average pre-pause heart rate are compared to their respective thresholds. All the threshold values are programmable and can be adjusted by the user. If the trigger noise count is less than the noise counter threshold NC_{TH}, and if the post-pause amplitude ratio is less than the post-pause amplitude threshold POSTR_{TH}, and if the average pre-pause heart rate is greater than or equal to the heart rate threshold PHR_{TH}, then at 615 the pause is rejected, that is, the device-detected pause is deemed a “false positive” pause detection. Otherwise, the pause is verified at 616, that is, the device-detected pause is deemed a “true positive” pause detection.

The Shannon entropy-based detector 425 can verify a presence or absence of cardiac pause using certain metrics generated by the feature extraction circuit 422, including the Shannon entropy of inner pause segment 528 and pre-pause R amplitude 521. FIG. 6B is a flow chart of an example pause verification algorithm 600B that may be used by the Shannon entropy-based detector 425. At 621, the Shannon entropy of inner pause segment, optionally along with other signal metrics, can be obtained from the feature extraction circuit 422. At 622, the Shannon entropy can be compared a first entropy threshold value H_{TH1}. If the Shannon entropy is greater than or equal to H_{TH1}, then the pause is rejected at 624, that is, the device-detected pause is deemed a “false positive” pause detection. If at 622 the Shannon entropy is less than H_{TH1}, then Shannon entropy can be compared to a second entropy threshold H_{TH2} smaller than the first threshold H_{TH1} at 623. The pre-pause R amplitude may also be checked against a corresponding R wave amplitude threshold PRAMP_{TH} at 623. If Shannon entropy is greater than or equal to H_{TH2}, and if the pre-pause R amplitude is less than PRAMP_{TH}, then the pause is rejected at 624, that is, the device-detected pause is deemed a “false positive” pause detection. Otherwise, the pause is verified at 625, that is, the device-detected pause is deemed a “true positive” pause detection.

The sample entropy-based detector 426 can verify a presence or absence of cardiac pause using certain metrics generated by the feature extraction circuit 422, including the sample entropy of inner pause segment 529 and pre-pause amplitude ratio 524. FIG. 6C is a flow chart of an example pause verification algorithm 600C that may be used by the sample entropy-based detector 426. At 631, the sample entropy of inner pause segment, optionally along with other signal metrics, can be obtained from the feature extraction circuit 422. At 632, the sample entropy and the pre-pause amplitude ratio can be compared to respective thresholds. If the sample entropy is less than the sample entropy threshold SampEN_{TH}, and if the pre-pause amplitude ratio is less than a corresponding ratio threshold POSTR_{TH}, then the pause is rejected at 633, that is, the device-detected pause is deemed a “false positive” pause detection. Otherwise, the pause is verified at 634, that is, the device-detected pause is deemed a “true positive” pause detection.

The enhanced R-peak based detector 427 can verify a presence or absence of cardiac pause based on an enhanced R wave detection algorithm different from and more sensitive than the R wave detection algorithm used in the medical device as the basis for detecting a cardiac pause. As described above with respect to FIGS. 2 and 3A-3B, one of the causes of false positive detection of cardiac pause episodes is undersensing of R waves due to (physiological) R wave amplitude drop. The enhanced R wave detection algorithm used by the enhanced R-peak based detector 427 can reduce the likelihood of R wave undersensing, and thereby improving cardiac pause detection. In an example, the enhanced R-peak based detector 427 may use Pan-Tompkins algorithm to detect R waves from the ECG signal recorded by the medical device, As described in “A Real-Time QRS Detection Algorithm,” IEEE Transactions on Biomedical Engineering. BME-32 (3): 230-236, March 1995, the Pan-Tompkins algorithm applies a series of filters to highlight the frequency content of this rapid heart depolarization and removes the background noise. Then, it squares the signal to amplify the QRS contribution, which makes identifying the QRS complex more straightforward. Finally, it applies adaptive thresholds to detect the peaks of the filtered signal.

FIG. 6D is a flow chart of an example pause verification algorithm 600D that may be used by the enhanced R-peak based detector 427. At 641, an enhanced R wave detection algorithm, such as Pan-Tompkins algorithms, may be used to detect R waves in the device-detected cardiac signal (e.g., ECG). At 642, information about device-detected and stored R waves, including R wave timings, can be obtained from the stored event markers 414. At 643, a difference between a count of R waves detected using the enhanced detection algorithm and a count of device-detected R waves in the cardiac signal can be computed. At 644, the difference of R wave counts can be compared to a threshold RC_{TH}. If the difference exceeds the threshold RC_{TH}, then the pause is rejected at 645, that is, the device-detected pause is deemed a “false positive” pause detection. Otherwise, the pause is verified at 646, that is, the device-detected pause is deemed a “true positive” pause detection.

In some examples, two or more of the detectors selected from detectors 424, 425, 426, and 427 may be combined to verify a presence or absence of cardiac pause in the device-detected cardiac pause episode. The combination can be a logic combination. In an example, the detectors 424, 425, 426, and 427 can be combined using “AND” logic to form a composite pause detection algorithm 600E, as illustrated in FIG. 6E. The detectors 424, 425, 426, and 427 may each perform their respective pause verification algorithms 651, 652, 653, and 654 as described above in FIGS. 6A-6D. Cardiac pause is verified to present in the device-detected cardiac pause episode at 656 (that is, the device-detected pause episode is a “true positive” pause event) only if each and every detector of detectors 424, 425, 426, and 427 verify the presence cardiac pause. If any one of the 424, 425, 426, or 427 rejects the pause (i.e., absence of pause in the device-detected cardiac pause episode), then it can be determined at 655 that no pause is present (that is, the device-detected pause episode is a “false positive” pause event). Such logic combination of multiple detectors can ensure a high accuracy of detecting real cardiac pause events. The order of employing the detectors 424, 425, 426, and 427 as shown in FIG. 6E is by way of example and not limitation. Other orders may be used. In some examples, different logic combinations of any or all of the detectors 424, 425, 426, and 427 may be used.

The user interface 430 may include an input device and an output device. The input device may receive a user's programming input, such as parameters and threshold values used by the feature extraction circuit 422 to generate signal metrics, or by the cardiac pause verification circuit 423 (or any of the detectors therein) for verifying the presence or absence of pause in the device-detected cardiac pause episode. The input device may include a keyboard, on-screen keyboard, mouse, trackball, touchpad, touchscreen, or other pointing or navigating devices. The output device may generate a human-perceptible presentation of the verification of the presence or absence of the pause in the device-detected cardiac pause episode. The output device may include a display for displaying the device-detected pause episode (including, for example, the cardiac signal and event markers), and any intermediate measurements or computations produced by the episode segmentation circuit 421, the feature extraction circuit 422, or the cardiac pause verification circuit 423. In an example, the output device may automatically display the information about the cardiac pause episode in response to the verified presence of cardiac pause in the device-detected cardiac pause episodes, and not to automatically display he information about the cardiac pause episode if cardiac pause is not verified in the device-detected cardiac pause episodes, but may be displayed in a commanded mode upon a user's request. In some examples, the processor circuit 420 may determine a risk of syncope in the patient based at least in part on the verified presence or absence of the cardiac pause. The output device may generate an alert to the user about the patient risk of syncope. The output unit may include a printer for printing hard copies of the detection information. The information may be presented in a table, a chart, a diagram, or any other types of textual, tabular, or graphical presentation formats. The presentation of the output information may include audio or other media format. The output unit may generate an alert to the system user or a recommendation for reviewing the verified pause episodes, or for initiating or adjusting treatment.

The optional therapy controller 440 may be configured to provide a control signal to initiate or adjust a therapy to the patient in response to the verification of the presence of pause episode. Examples of the therapy may include electrostimulation therapy delivered to the heart, a nerve tissue, other target tissues, a cardioversion therapy, a defibrillation therapy, or drug therapy. In some examples, the therapy controller 440 may modify an existing therapy, such as adjust a stimulation parameter or drug dosage.

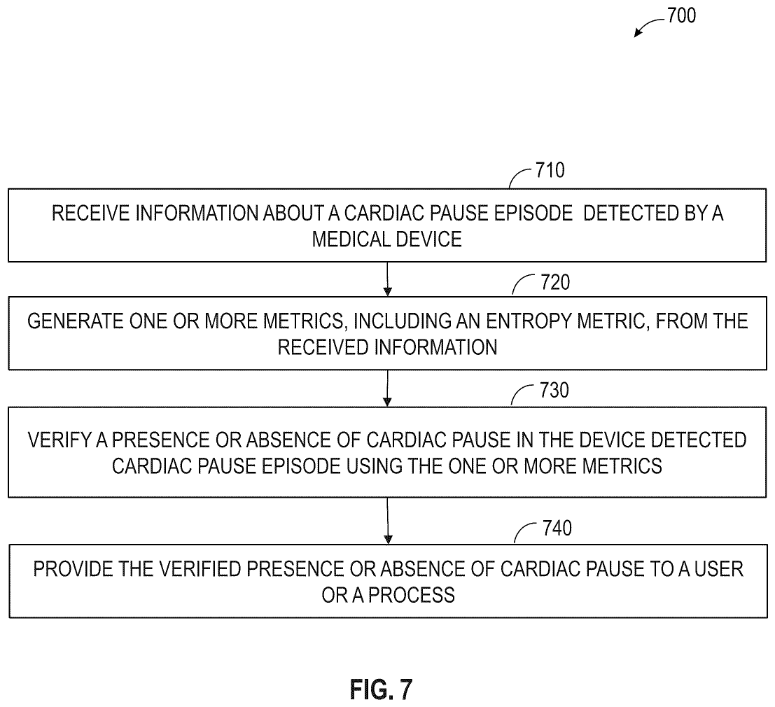

FIG. 7 is a flowchart illustrating an example of a method 700 of verifying cardiac pause episodes detected by a medical device in a patient. As described above, due to the constraints of battery power and computational resources in an implantable or wearable medical device, simple device-based pause detection algorithms may cause inappropriately detected, or false positive, pause episodes. The method 700, which may be implemented in and executed using the cardiac pause verification system 400, can identify which device-detected pause episodes are true positive episodes (indicating the device correctly detects the cardiac pause events) and which are false positive episodes (indicating the device falsely detects the cardiac pause events).

The method 700 begins at step 710, where information about a cardiac pause episode detected by a medical device can be received. The medical device detects the cardiac pause episode using a device-based detection algorithm. The cardiac pause episode information may include a physiological signal sensed in response to the detection of a cardiac pause event. Examples of the physiologic signal may include cardiac signals such as surface electrocardiogramaubcutaneous ECG, or intracardiac electrogram (EGM), among other physiological signals that may be sensed via one or more implantable, wearable, or otherwise ambulatory sensors or electrodes associated with the patient. The cardiac pause episode information may further include event markers that identify the type or timing of certain events, including a pause onset marker and pause end marker (“pause start”) marking respectively the beginning (onset) and the end of the device-detected cardiac pause episode, type or timings of heart beats or noise events in the cardiac signal recognized by the medical device, among others.

At 720, one or more metrics may be generated from the received cardiac pause episode information. In an example where the cardiac pause episode information includes a cardia signal (e.g., an ECG), the signal metrics may be generated from one or more segments of the cardiac signal. In an example, the cardiac signal may be partitioned a pre-pause segment defined as the segment of the cardiac signal before a pause onset marker, a post-pause segment defined as the segment after a pause end marker, and an inner pause segment defined as the portion of the cardiac signal between the pause onset marker and the pause end marker. By ways of example and not limitation and as described above with respect to FIG. 5, the signal metrics may include one or more of a pre-pause R amplitude defined as an average amplitude of a specific number of R-waves before the pause onset marker, a post-pause R amplitude defined as an average amplitude of a specific number of R-waves after the pause end marker, a representative inner pause signal amplitude defined as a specific percentile (e.g., 98th percentile) signal amplitude of the absolute of amplitudes present in the inner pause segment, a pre-pause amplitude ratio defined as a ratio of a representative cardiac event amplitude in the pre-pause segment to the representative inner pause signal amplitude, a post-pause amplitude ratio defined as a ratio of a representative cardiac event amplitude in the post-pause segment to the representative inner pause signal amplitude, an average pre-pause heart rate defined as the average heart rate using the device-detected heart beats (e.g., R waves) in the pre-pause segment and stored as event timing markers in the medical device, an average post-pause heart rate defined as the average heart rate using the device-detected heart beats (e.g., R waves) in the post-pause segment, a Shannon entropy of the inner pause segment, or a Sample entropy of the inner pause segment, among others.

At 730, whether or not a cardiac pause is present in the device-detected cardiac pause episode may be verified, such as using the cardiac pause verification circuit 423. A different algorithm different from the device-based detection algorithm may be used to in the verification process. Examples of the verification algorithms may include a trigger noise counter-based detector, a Shannon entropy-based detector, a sample entropy-based detector, or an enhanced R-peak based detector, as described above with respect to FIG. 4. Each of these detectors is capable of detecting or verifying a presence or absence of cardiac pause using one or more signal metrics, such as those described above with respect to FIG. 5. Examples of methods for verifying the presence or absence of cardiac pause in a device-detected cardiac pause episode using one or more signal metrics are described above with reference to FIGS. 6A-6D. In some examples, two or more of the detectors as described above may be combined in the verification process. The combination can be a logic combination, such as an “AND” logic of a number of detectors as described above with respect to FIG. 6E.

At 740, the verification of the presence or absence of the pause in the device-detected cardiac pause episode may be provided to a user or a process, such as via an output device of the user interface 430. In an example, the output device may automatically display the information about the cardiac pause episode in response to the verified presence of cardiac pause in the device-detected cardiac pause episodes, and not to automatically display he information about the cardiac pause episode if cardiac pause is not verified in the device-detected cardiac pause episodes, but may be displayed in a commanded mode upon a user's request. In some examples, a risk of syncope may be determined for the patient based at least in part on the verified presence or absence of the cardiac pause. An alert about the patient risk of syncope may be provide to the user. In some examples, in response to the verification of the presence of pause episode, a therapy (e.g., drug therapy or electrostimulation therapy) may be initiated or adjusted to treat cardiac pause or to prevent syncope.

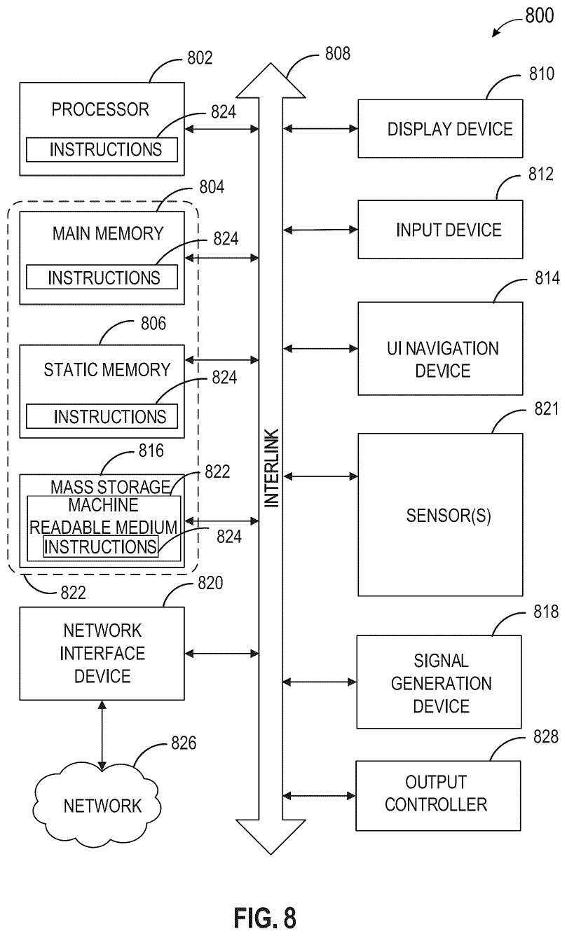

FIG. 8 illustrates generally a block diagram of an example machine 800 upon which any one or more of the techniques (e.g., methodologies) discussed herein may perform. Portions of this description may apply to the computing framework of various portions of the patient management system 100 or of the cardiac pause verification system 400.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810 (e.g., a raster display, vector display, holographic display, etc.), an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensors. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine-readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine-readable media.

While the machine-readable medium 822 is illustrated as a single medium, the term “machine-readable medium” may include a single medium or multiple media (e.g., a centralized or distributed database, and/or associated caches and servers) configured to store the one or more instructions 824.

The term “machine-readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802. 16 family of standards known as WiMax®), IEEE 802. 15. 4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical-device system for verifying cardiac pause episodes detected by a medical device in a patient, the system comprising:
a cardiac pause verification circuit configured to:
receive information about a device-detected cardiac pause episode detected by the medical device using a device-based detection algorithm;
generate one or more metrics, including at least one entropy metric, from the received information about the device-detected cardiac pause episode; and
verify a presence or absence of cardiac pause in the device-detected cardiac pause episode using the generated one or more metrics and a verification algorithm different from the device-based detection algorithm; and
an output circuit configured to provide the verified presence or absence of the cardiac pause to a user or a process executable by the medical-device system.

2. The medical-device system of claim 1, wherein the received information about the device-detected cardiac pause episode includes a cardiac signal and event markers generated by and stored in the medical device.

3. The medical-device system of claim 2, wherein the event markers include a pause onset marker marking a beginning of the device-detected cardiac pause episode, and a pause end marker marking an end of the device-detected cardiac pause episode, the pause onset marker and the pause end marker partitioning the cardiac signal into a pre-pause segment prior to the pause onset marker, an inner pause segment between the pause onset marker and the pause end marker, and a post-pause segment following the pause end marker.

4. The medical-device system of claim 3, wherein the event markers further include type or timings of heart beats or noise events in the cardiac signal recognized by the medical device.

5. The medical-device system of claim 3, wherein the at least one entropy metric includes a Shannon entropy of the inner pause segment of the cardiac signal, the one or more metrics include a representative amplitude of cardiac events in the pre-pause segment,
wherein the cardiac pause verification circuit is configured to verify the presence or absence of cardiac pause in the device-detected cardiac pause episode using the Shannon entropy of the inner pause segment and the representative amplitude of cardiac events in the pre-pause segment.

6. The medical-device system of claim 3, wherein the at least one entropy metric includes a sample entropy of the inner pause segment of the cardiac signal, the one or more metrics include a first amplitude ratio of a representative cardiac event amplitude in the pre-pause segment to a representative amplitude of the inner pause segment,
wherein the cardiac pause verification circuit is configured to verify the presence or absence of cardiac pause in the device-detected cardiac pause episode using the sample entropy of the inner pause segment and the first amplitude ratio.

7. The medical-device system of claim 3, wherein the cardiac pause verification circuit is configured to verify the presence or absence of cardiac pause in the device-detected cardiac pause episode further using one or more of:
a count of noise events detected by the medical device from a specific portion of the pre-pause segment;
an average heart rate in the pre-pause segment; or
a second amplitude ratio of a representative amplitude of cardiac events in the post-pause segment to a representative amplitude of the inner pause segment.

8. The medical-device system of claim 3, wherein the cardiac signal includes an electrocardiogram (ECG), the device-based detection algorithm includes a first R wave detector, the verification algorithm includes a second R wave detector different from and more sensitive than the first R wave detector in detecting R wave timings in the ECG,
wherein the cardiac pause verification circuit is configured to verify the presence or absence of cardiac pause in the device-detected cardiac pause episode further based on a comparison timings of R waves detected by the second R wave detector and timings of R waves detected by the first R wave detector.

9. The medical-device system of claim 1, wherein the verification algorithm includes a decision tree taking the one or more metrics as decision nodes, or neural network model taking the one or more metrics as input layer parameters.

10. The medical-device system of claim 1, comprising an ambulatory medical device (AMD) and an external system operatively in communication with the AMD,
wherein the AMD includes circuitry configured to sense physiological information in a patient, to detect the cardiac pause episode using the sensed physiological information, and to store the information about the detected cardiac pause episode,
wherein the external system including the cardiac pause verification circuit.

11. The medical-device system of claim 1, wherein the output circuit is configured to:
present the information about the device-detected cardiac pause episode on a display in response to the verified presence of cardiac pause in the device-detected cardiac pause episodes; and
withhold display of the information about the device-detected cardiac pause episode in response to the verified absence of cardiac pause in the device-detected cardiac pause episodes.

12. The medical-device system of claim 1, comprising an event detector circuit configured to determine a risk of syncope in the patient based at least in part on the verified presence or absence of the cardiac pause.

13. A method of verifying cardiac pause episodes detected by a medical device in a patient, the method comprising:
receiving information about a device-detected cardiac pause episode detected by the medical device using a device-based detection algorithm;
generating one or more metrics, including at least one entropy metric, from the received information about the device-detected cardiac pause episode;
verifying a presence or absence of cardiac pause in the device-detected cardiac pause episode using the generated one or more metrics and a verification algorithm different from the device-based detection algorithm; and
providing the verified presence or absence of the cardiac pause to a user or a process.

14. The method of claim 13, wherein the received information about the device-detected cardiac pause episode includes a cardiac signal and event markers generated by and stored in the medical device.

15. The method of claim 14, wherein the event markers include a pause onset marker marking a beginning of the device-detected cardiac pause episode, and a pause end marker marking an end of the device-detected cardiac pause episode, the pause onset marker and the pause end marker partitioning the cardiac signal into a pre-pause segment prior to the pause onset marker, an inner pause segment between the pause onset marker and the pause end marker, and a post-pause segment following the pause end marker.

16. The method of claim 15, wherein the at least one entropy metric includes a Shannon entropy of the inner pause segment of the cardiac signal, the one or more metrics include a representative amplitude of cardiac events in the pre-pause segment, wherein verifying the presence or absence of cardiac pause in the device-detected cardiac pause episode includes using the Shannon entropy of the inner pause segment and the representative amplitude of cardiac events in the pre-pause segment.

17. The method of claim 15, wherein the at least one entropy metric includes a sample entropy of the inner pause segment of the cardiac signal, the one or more metrics include a first amplitude ratio of a representative cardiac event amplitude in the pre-pause segment to a representative amplitude of the inner pause segment,
wherein verifying the presence or absence of cardiac pause in the device-detected cardiac pause episode includes using the sample entropy of the inner pause segment and the first amplitude ratio.

18. The method of claim 15, wherein verifying the presence or absence of cardiac pause in the device-detected cardiac pause episode includes using one or more of:
a count of noise events detected by the medical device from a specific portion of the pre-pause segment;
an average heart rate in the pre-pause segment; or
a second amplitude ratio of a representative amplitude of cardiac events in the post-pause segment to a representative amplitude of the inner pause segment.

19. The method of claim 15, wherein the cardiac signal includes an electrocardiogram (ECG), the device-based detection algorithm includes a first R wave detector, the verification algorithm includes a second R wave detector different from and more sensitive than the first R wave detector in detecting R wave timings in the ECG,
wherein verifying the presence or absence of cardiac pause in the device-detected cardiac pause episode is based at least in part on a comparison of timings of R waves detected by the second R wave detector and timings of R waves detected by the first R wave detector.

20. The method of claim 13, comprising:
presenting the information about the device-detected cardiac pause episode on a display in response to the verified presence of cardiac pause in the device-detected cardiac pause episodes; and
withholding display of the information about the device-detected cardiac pause episode in response to the verified absence of cardiac pause in the device-detected cardiac pause episodes.
